(19) United States
(12) Patent Application Publication
(10) Pub. No.: US 2025/0176842 A1
(43) Pub. Date: Jun. 5, 2025

(54) PORTABLE PHYSIOLOGICAL MEASUREMENT DEVICE

(71) Applicant: Withings, ISSY LES MOULINEAUX (FR)

(72) Inventors: Thomas BEDETTI, ISSY LES MOULINEAUX (FR); Antoine MERLOT, ISSY LES MOULINEAUX (FR); Benoit TUCOULAT, ISSY LES MOULINEAUX (FR); Thibaut BARBEDETTE, ISSY LES MOULINEAUX (FR); Thomas DROZE, ISSY LES MOULINEAUX (FR)

(21) Appl. No.: 18/969,711

(22) Filed: Dec. 5, 2024

(30) Foreign Application Priority Data
Dec. 5, 2023 (FR) 2313608

(51) Int. Cl. A61B 5/0205; A61B 5/00; A61B 5/024; A61B 5/26; A61B 5/332; A61B 7/04

(52) U.S. Cl. CPC A61B 5/02055; A61B 5/02438; A61B 5/26; A61B 5/7445; A61B 5/02416; A61B 5/332; A61B 7/04

(57) ABSTRACT

A portable physiological measurement device holdable by a user, includes a housing of elongated shape along a direction of extension (X) and including, along the direction of extension, a first end and a second end, a first physiological end sensor positioned at the first end, a second physiological end sensor positioned at the second end, a physiological finger sensor arranged on the housing near the first end or the second end.

### CROSS-REFERENCE TO RELATED APPLICATIONS

This application claims priority to French Patent Application No. 2313608, filed Dec. 5, 2023, the entire content of which is incorporated herein by reference in its entirety.

### FIELD

The present invention relates to portable physiological measurement devices (hereinafter referred to as the measurement device) of the personal hand-held monitor (PHHM) type, incorporating a plurality of physiological data sensors.

### BACKGROUND

Numerous measuring devices exist. Some are capable of performing a single measurement: thermometer, stethoscope, ECG, etc., while others are capable of performing several such measurements.

Electronic stethoscopes include those described in the following documents: U.S. Pat. No. 9,265,478, US20220354451, U.S. Ser. No. 11/741,931, US20220031256, US20010030077, US20230142937, U.S. Pat. Nos. 5,737,429A, 5,638,453A. However, not all of these documents provide devices suitable for self-measurement, and some are even explicitly designed to be held by a physician. In addition, the number of measurements may be insufficient to have a device suitable for RPM.

Thermometers include the one described in WO2017114923A2 (on behalf of Withings™).

US2022035445 in the name of EKO HEALTH™ and more generally all publications by this company describe an electronic stethoscope incorporating an electrocardiogram.

Other documents describe devices capable of two or three measurements.

For example, document PL423977A1 describes a stethoscope with an infrared sensor, but without detailing the integration.

For example, WO2022200743 describes a device with a parallelepiped housing that integrates a stethoscope, an oximeter, an otoscope and a thermometer, in addition to a cuff for pressure measurement.

For example, US20200015774 describes a device with a circular handle for bringing a base into contact with a user. The base comprises an electronic stethoscope which may also include a thermometer or oximeter. The disclosure also mentions, without any details, ECG and ultrasound signal capture.

For example, WO201704463 describes a finger-worn device for oximetry that integrates a stethoscope and an otoscope.

For example, document WO2022253723 describes an essentially cylindrical device with two opposing faces comprising three physiological sensors: thermometer, stethoscope and oximeter.

For example, the Linktop 6-in-1™ product offers a device in the shape of a square mini-slab, featuring a temperature sensor and an electrode on the same side face, an optical sensor and an electrode on the square upper face, and an electrode on the square lower face.

Nevertheless, these documents have their limitations. One limitation lies in the transition from a conceptual idea to a functional product on the market. Another limitation lies in the number of measurements that can be performed by each device: one or two measurements, rarely three. To have an effective RPM device, it is paramount to maximize the number of measurements on a single device. Another limitation lies in ergonomics and ease of use, especially as the number of available measurements increases. Indeed, the measurement device must remain small and easy to handle by users who may be elderly and in poor health. These technical constraints have a direct impact on user adoption and retention.

### SUMMARY

One or more aspects of the present invention relate to a portable physiological measurement device (hereinafter referred to as the measurement device) of the personal hand-held monitor (PHHM) type, incorporating a plurality of physiological data sensors. Such a device can be used for remote monitoring, for example by a physician during a teleconsultation or asynchronous consultation. The term “telehealth” or “remote patient monitoring” (RPM) is more commonly used. For the purposes of this description, the term RPM will be used.

One of the features of the disclosed device is that it may be operated in self-measurement mode, i.e. the person holding the device is the person on whom the measurements are taken.

The measurements taken are physiological measurements of a user holding the measuring device in his hand. These measurements are representative of the user's health status.

An aspect of the disclosure relates to measuring devices offering improved ease of use. This may relate to ergonomics. It may concern the number of measurements the device can perform.

More particularly, various aspects of the invention are defined in the claims.

According to one aspect of the present description, there is presented a portable physiological measurement device holdable by a manipulator or user comprising:
a housing of elongated shape along an extension direction and comprising, along the direction of extension, a first end defining a first edge and a second end defining a second edge,
a first physiological end sensor positioned at the first end and comprising a functional surface intended to be positioned facing a user, the functional surface being inscribed in the edge at the end,
a second physiological end sensor positioned at the second end,
a physiological finger sensor arranged on the housing near the first end or the second end.

This device is easy to handle by a user who is himself the manipulator. Thanks to the inscribed nature of the first end sensor, it does not interfere with holding, either with one or two hands.

More precisely, the device is configured so that when the device is held by a hand on the side of the first end, in the extension of the longitudinal direction, the physiological sensor does not interfere and wherein the physiological finger sensor is positioned so that when gripping a finger of the hand can be in contact with the finger sensor.

In particular, the physiological finger sensor is arranged on the housing to receive an index finger of one hand and/or a thumb of one hand.

In an embodiment, the second end defines a second edge and the second physiological sensor comprises a functional surface inscribed in the second edge. The result is a symmetrical operating device for the end sensors, which does not interfere with one-handed (in either direction) and two-handed gripping.

In an embodiment, the device comprises an essentially parallelepiped shape with a front face and a rear face arranged between the first end and the second end. The second physiological end sensor is arranged on the rear face, near the second end.

In an embodiment, the distance along the extension direction between the first end and the second end is of length L and “arranged near the first end (or second end)” means “between the first edge (or second edge) and strictly half the length L from the first edge (or second edge), or even strictly a quarter of the length L from the first edge (or second edge).

In an embodiment, the physiological end sensor, and in particular the functional surface of the physiological end sensor, is positioned within the volume defined by the housing or, in one embodiment, no more than 1 mm outside the volume defined by the housing. This ensures that the end sensor does not interfere with the gripping of the device.

The physiological end sensor may be an audio sensor (e.g. piezoelectric) and the functional surface may comprise a membrane. The physiological end sensor may be a temperature sensor and the functional surface may comprise a cone. The physiological end sensor may be a spirometer and the functional surface may comprise a mouthpiece.

In an embodiment, the functional surface is orthogonal to the direction of extension at the end and/or the side face defined by the first edge (and/or the second edge) is orthogonal to the direction of extension at the end.

In an embodiment, the physiological finger sensor comprises an optical sensor, for example a PPG-type sensor with LEDs or a laser. In particular, the optical sensor can be used to measure heart rate and its variations, or oxygen saturation.

In an embodiment, the physiological finger sensor is a first physiological finger sensor, arranged on the housing near the first end, and the device further comprises a second physiological finger sensor, arranged on the housing near the second end.

For example, the first and/or second physiological finger sensor comprises an electrode. For example, the electrodes are electrocardiogram (ECG) electrodes. More particularly, there may be only two ECG electrodes to perform an ECG. This arrangement simplifies the handling of an ECG, since only two electrodes need to be touched, rather than three or more. Alternatively or additionally, the electrodes can be impedance measurement electrodes, for impedance analysis.

In an embodiment, the first physiological finger sensor and the second physiological finger sensor are aligned parallel to the extension direction.

In an embodiment, the device comprises a physical interface, for example a navigation button. The physical interface is then arranged on the housing and the second physiological finger sensor can be positioned on the physical interface.

In an embodiment, the device comprises an essentially parallelepiped shape with a front face and an upper face arranged between the first end and the second end, the front face and the upper face being connected, for example perpendicularly to each other.

In an embodiment, the first physiological finger sensor and/or the second physiological finger sensor are positioned on the upper face.

In an embodiment, the device comprises a display.

The display can be located on the front face.

In an embodiment: in a one-handed handling position for the first end sensor, the display is configured to display information along a reading direction transverse to the extension direction; in a two-handed handling position in which the finger sensor is used, the display is configured to display information along a reading direction parallel to the extension direction.

The device can include a gyrometer and/an accelerometer configured to determine the spatial orientation of the device and to adapt the reading direction of the display in accordance with this orientation.

In an embodiment, the device comprises a physical interface, such as a navigation button, positioned on the front face. The physical interface is positioned between the second edge and half, or even a third, of a length of the housing along the direction of extension from the second edge. Alternatively, the physical interface is positioned between the first edge and half, or even a third, of a length of the housing along the direction of extension from the first edge.

In an embodiment, the finger sensor is positioned within the volume defined by the housing, or a maximum of 2 mm outside the volume defined by the housing.

According to another aspect of the present description, a method of using a device as previously described is presented. The method may comprise the following steps:
measurement with the physiological end sensor by the user in a one-handed handling position,
measurement with the finger physiological sensor by the user in a two-handed handling position.

The process can also include measurement with the second physiological sensor by the user in a one-handed handling position,

In one embodiment, wherein the device comprises a display and a physical interface, the method further comprises a step of user selection of the measurement to be performed on the display with the physical interface in a navigation position. The navigation position is typically one-handed.

### BRIEF DESCRIPTION OF THE DRAWINGS

Further features, details and benefits will become apparent from the detailed description below, and from an analysis of the appended drawings, on which:

FIG. 1 This figure shows two 3D views, right and left, of a measuring device in an embodiment.

FIG. 2 This figure shows four projected views of the device shown in FIG. 1.

FIG. 3 This figure shows a view of the device shown in FIGS. 1 to 2, when operated in the navigation position with one hand.

FIG. 4 This figure shows a view of the device shown in FIGS. 1 to 3, during a handling in a one-handed position for a first sensor of the device.

FIG. 5 This figure shows a view of the device shown in FIGS. 1 to 4, during a handling in a one-handed position, for a second sensor of the device.

FIG. 6 This figure shows a view of the device shown in FIGS. 1 to 5, when a finger sensor of the device is operated in a two-handed position.

FIG. 7 This figure shows two 3D views, right and left, of a measuring device in another embodiment.

FIG. 8 This figure shows a view of the device shown in FIG. 7, when operated in the navigation position with one hand.

FIG. 9 This figure shows a view of the device shown in FIGS. 7 to 8, when a second sensor in the device is handled in a one-handed position.

FIG. 10 This figure shows two projected views, front face and rear face, of a measuring device in another embodiment.

FIG. 11 This figure shows a schematic view of the devices and their environment.

### DETAILED DESCRIPTION

### General Presentation

The present description will describe several embodiments and variants of physiological measurement devices that are portable, holdable by one or two hands and suitable for self-measurement. The term “device” will be used to simplify the language. By definition, the manipulator is the one holding the device and the user is the one undergoing the measurement. For the purposes of this description, unless otherwise stated, the manipulator and the user are considered to be one and the same.

The device incorporates one or more physiological sensors for measuring physiological characteristics (“physiological measurement”) of a user who is also the manipulator. In this respect, the sensor may be one of: a temperature sensor to measure the user's body temperature, a sound sensor to measure heart or lung sounds, an ECG (electrocardiogram) sensor to measure heartbeat and rhythm, an impedance sensor, an optical sensor of the PPG (PhotoPlethysmoGraphy) type, for example, for oximetry, to measure blood oxygen saturation (SpO2) or other quantities such as heart rate, a force sensor, to measure relative force or pressure on the finger (ultimately to determine arterial pressure), a spirometer, and so on.

By physiological measurement, it is meant measurements of a physiological characteristic of a user (hereinafter referred to as a user), which reflect a health status, such as: temperature, heart sounds, lung sounds, heart rate, arrhythmia, etc. In particular, in an embodiment, the device incorporates at least two physiological sensors; in another embodiment, the device incorporates at least three physiological sensors (e.g. thermometer, stethoscope, ECG); in yet another embodiment, the device incorporates at least four physiological sensors (e.g. thermometer, stethoscope, ECG, PPG).

By portable, it is meant a device that is lightweight and space-saving. By lightweight, it is meant a device that weighs less than 300 g. The device can therefore be easily handled with one hand by a user and the device can, for example, be easily stored in a drawer, handbag or trouser pocket. For example, the device weighs less than 250 g, or even 150 g. By way of example, the device has a volume of less than 20×5×10 cm, or even 18×3×5 cm, or even 15×2.5×4 cm.

The device has an elongated shape in one extension direction, for example an essentially parallelepiped shape. By “essentially parallelepiped shape,” it is meant that the device approximates a rectangular prism, with sides that are predominantly flat or slightly curved to facilitate ergonomic handling. This definition allows for minor variations, such as rounded edges or corners, to enhance user comfort and safety during manipulation, provided these variations do not significantly alter the overall geometric resemblance to a parallelepiped. Such adjustments are consistent with the device's design intent for portability and ease of use.

In addition, the device may be connected, in the sense that it may send data to a third-party device, such as a smartphone or server. This connectivity enables the device to function as an RPM device, with the user becoming a patient of a remote physician. Teleconsultation may be synchronous, with live or quasi-direct interaction with the physician, or asynchronous. In synchronous mode, the patient uses the device to acquire physiological data, which are immediately or almost immediately transmitted to the physician (a few seconds later). In asynchronous mode, the patient uses the device when he or she can, and the physician consults the physiological data when he or she can, i.e. later in time.

The shape of the housing and the arrangement of the sensor(s) in the device is designed to make it easy to use with one hand to perform certain physiological measurements (e.g. temperature and heart or lung sound) and with two hands to perform other physiological measurements (e.g. ECG or PPG). The device is structured to allow one-handed operation for measurements such as temperature and heart or lung sounds, ensuring accessibility even for users with limited dexterity or strength. For more complex measurements, such as ECG or PPG, the device is configured for two-handed use, providing greater stability and precision during operation. In an embodiment, the one-handed position may resemble a remote control position, with which users are generally familiar, and the two-handed position may resemble a video game console controller position, with which users are also generally familiar. These configurations not only optimize the functional interaction with the device but also contribute to user confidence, particularly among elderly individuals or those with limited experience handling medical devices. By combining ergonomic design with sensor placement tailored to natural hand positions, the device promotes effective and reliable physiological data collection.

The device is intended for self-measurement, empowering users to independently monitor their physiological data without requiring assistance. It must therefore be easy for the user to take measurements on his or her own. To achieve this, the device incorporates intuitive design elements that simplify operation, such as strategically placed sensors, a user-friendly interface, and ergonomic housing that supports natural handling. The ease of use ensures that users, including those who may have limited technical expertise or reduced mobility, can confidently and accurately take measurements on their own. Additionally, the self-measurement capability reduces dependence on healthcare professionals for routine monitoring, making the device particularly valuable for remote patient monitoring and telehealth applications. By combining accessibility with robust functionality, the device enhances user autonomy and promotes consistent health management.

While the device is optimized for self-measurement, the device may also be able to be used by two people, for example when one person is unable to handle the device (disability or child, for example). For instance, the device can be effectively operated by a caregiver or family member when the primary user, such as a person with a disability, limited dexterity, or a child, is unable to handle it independently. This dual-user adaptability ensures that the device remains functional and accessible in diverse contexts, including caregiving environments, pediatric settings, and situations involving patients with reduced motor control. By maintaining ergonomic flexibility and straightforward operational features, the device avoids imposing limitations on single-user functionality while simultaneously supporting seamless collaborative use. This design choice broadens the applicability of the device, enhancing its utility for individual and assisted health monitoring alike.

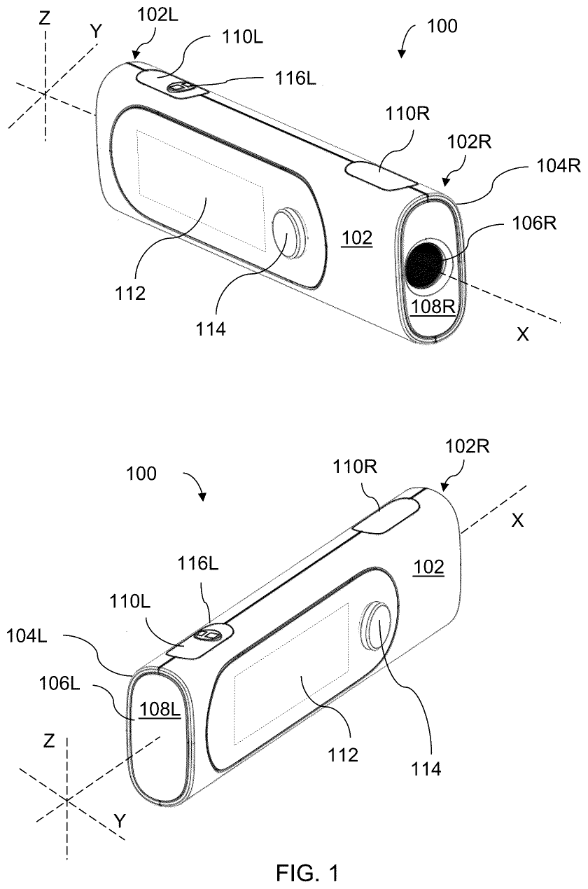

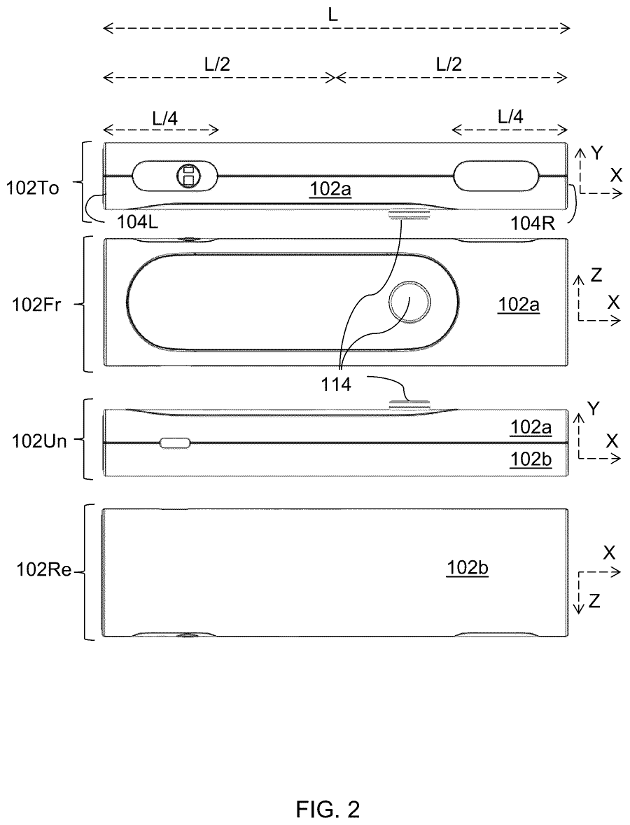

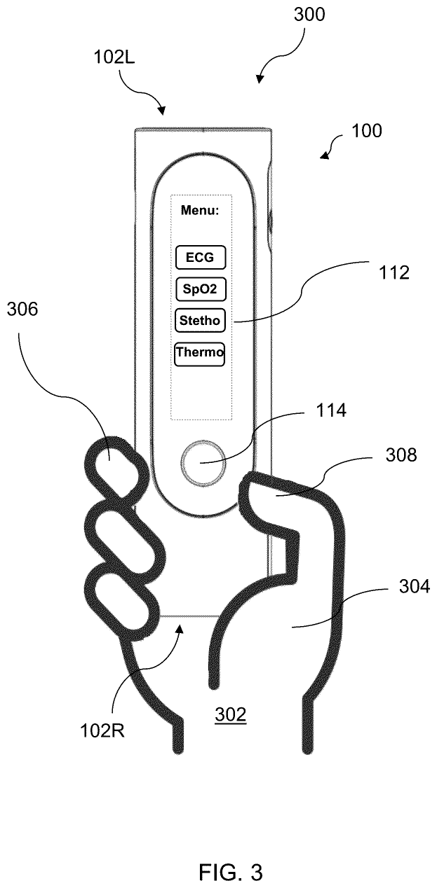

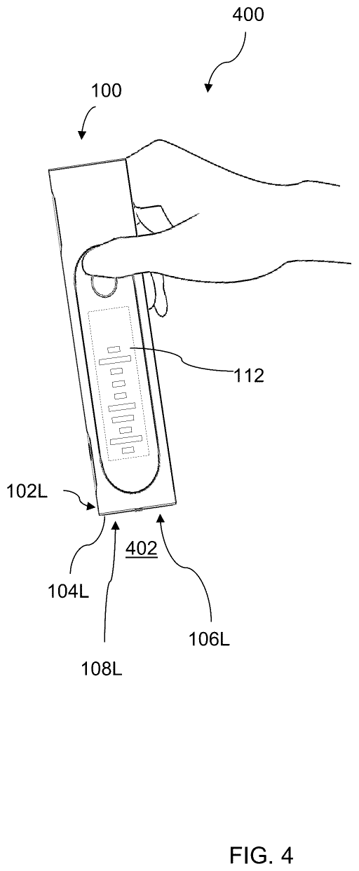

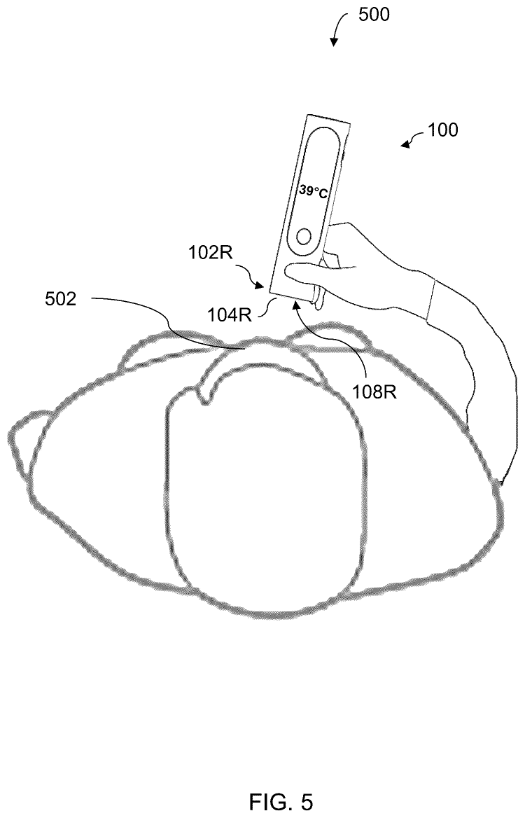

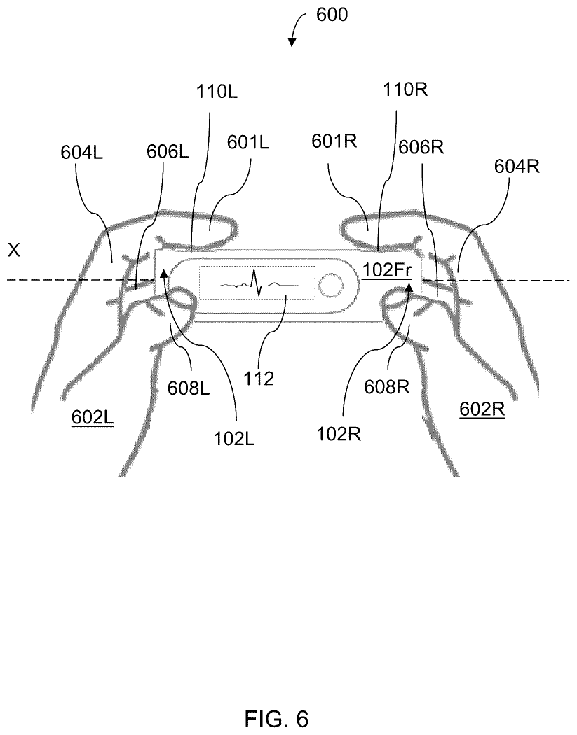

FIG. 1 shows two three-dimensional views of a device 100 in an embodiment. FIG. 2 shows the device 100 in four projections. FIG. 3 shows a view of device 100 in the navigation position, with one hand. FIGS. 4 and 5 each illustrate a view of device 100 when manipulated in a one-handed measurement position (for two different measurements). FIG. 6 illustrates a view of device 100 when operated in a two-handed measuring position.

### The Housing

Device 100 comprises an elongated housing 102 which defines an extension direction X. The housing 102 also defines two orthogonal transverse directions Y and Z. Hereafter, “transverse” is defined in relation to the X direction. This extension direction X is referred to as the principal direction, as the device 100 has its largest dimension along this direction. The extension direction X is rectilinear in the figures, but curvature is possible as long as handling of the device is not significantly impaired.

Along the extension direction X, the housing 102 comprises a first end 102L or first distal end (L for left) and a second end 102R or second distal end (R for right), opposite the first end 102L. The first end 102L defines a first edge 104L and the second end 102R defines a second edge 104R. Each edge 104L, 104R defines a closed curve (ovoid in the figures, due to the cross-section of housing 102 at first end 102L and second end 102R).

To enable easy holding, each edge 104L, 104R has a length of less than 30 cm, or even less than 15 cm. The description will present in detail the different possible handling positions of the device 100 by a user.

In an embodiment, the distance L between the two edges 104L, 104R along the extension direction X is less than 20 cm, or even less than 15 cm. This distance L ensures that the device 100 is portable.

In an embodiment, the distance L between the two edges 104L, 104R along the extension direction X is greater than 8 cm, or even greater than 10 cm. This distance L ensures that the device can be held with one or two hands. Indeed, a device that is too small cannot be handled easily, especially by everyone.

The device 100 is designed to be held by at least one of the two ends 102L, 102R with the hand in the extension of the housing 102 in the longitudinal direction. In particular, a navigation position is defined, illustrated in FIG. 3.

The housing 102 may have an essentially cylindrical shape, with a convex cross-section in the YZ plane (i.e. orthogonal to the extension direction X). In an embodiment, this cross-section has two axes of symmetry, for example the Y and Z axes as shown in the figures. The cross-section is, for example, oblong, as shown in the figures, or rectangular (with more or less rounded corners), or circular.

By essentially cylindrical, it is meant that the section orthogonal to the extension direction X shows no significant dimensional variation (e.g. less than 20% variation in maximum diameter).

The housing 102 is typically made of plastic, to be lightweight, cost-effective and electrically insulating. When the user holds the device 100, his or her hand(s) are mostly in contact with the housing 102. The housing 102 may be made up of several parts assembled together. In FIGS. 1 and 2, the housing 102 comprises two shells, noted 102a, 102b respectively, which may be assembled at a junction parallel to the extension direction X. Alternatively, the housing 102 is made up of a single shell. Other types of assembly are also possible.

At least two faces connecting edge 104L to edge 102R may be defined for the housing 102. In the housing of an oblong or rectangular cross-section, a front face 102Fr (Fr for “Front”), a rear face 102Re (Re for “Rear”) (opposite the front face), an upper face 102To (To for “Top”), and a lower face 102Un (Un for “Under”), opposite the upper face, are defined for the device, as illustrated in FIG. 2; finally, the two edges 104L, 104R each define a lateral face. The front face 102Fr and the rear face 102Re are connected by the upper face 102To and the lower face 102Un. In particular, the front face 102F and the upper face 102T are perpendicular or essentially perpendicular to each other (disregarding any possible roundings as shown in FIG. 1).

These face names are defined in relation to the two-handed handling position illustrated in FIG. 6.

As shown in FIG. 2, each of the upper face 102To and the lower face 102Un extends mainly in a longitudinal plane XY. Each of the front face 102Fr and the rear face 102R extends mainly in a longitudinal plane XZ, orthogonal to the plane XY. Each of the two edges 104L, 104R extends mainly in a transverse plane XZ, i.e. the side faces are also, in the figures, orthogonal to the extension direction X. Rounded shapes for the front face 102Fr, the rear face 102Re, the upper face 102To and the lower face 102Un may be provided, as shown in the figures, to have no ridge or thus facilitate holding.

The front face 102Fr and the rear face 102Re are taller along the Z dimension than the depth of the rear face 102Re and the upper face 102To along the Y dimension. In other words, the housing 102 is taller than it is deep.

The front face 102Fr and the rear face 102Re have a greater length along the X dimension compared to their height along the Z dimension. In other words, the housing 102 is longer than it is tall.

The same size proportions apply to the device 100. The device 100 has a length (measured along the X dimension) that is greater than its height (measured along the Z dimension) which is, in turn, greater than its depth (measured along the Y dimension). For example, the length is at least three (3) times greater than the height and the height is 1.5 times greater than the depth. These dimensions correspond to the specified volume of the device (in terms of X-dimension, Y-dimension, Z-dimension).

The device 100 comprises one or more physiological sensors arranged at different locations. The physiological sensors perform physiological measurements that generate physiological data.

### Physiological Sensors at the End

In particular, in an embodiment, the device 100 comprises a physiological end sensor 106L at the first end 102L, referred to as the first end sensor 106L.

The first end sensor 106L comprises a functional surface 108L. By functional surface 108L, it is meant a surface intended to be positioned facing the user, to interact with the latter, with or without contact, for obtaining the physiological measurement by the first end sensor 106L.

For example, the first end sensor 106L may be an electronic stethoscope, with a piezoelectric sensor and a membrane designed to be positioned on the user. The membrane functions as an amplifier of mechanical waves generated by the heart or lungs. In this housing, the functional surface 108L comprises the membrane. In particular, the membrane is the part of the piezoelectric sensor visible to the user. It is typically constructed from a durable, acoustically efficient material, such as polymer composites or high-grade elastomers, chosen for their ability to vibrate in response to sound waves while minimizing distortion. The thickness of the membrane may be within a range of 1 mm to 3 mm, notably 2 mm. A thinner membrane may enhance the sensitivity to faint sounds, whereas a slightly thicker membrane improves durability and resistance to wear during repeated use.

For example, the first end sensor 106L may be a thermometer, with a thermopile-type sensor and a lens. The lens may be surrounded by a cone. In this housing, the functional surface 108L comprises the lens and, where appropriate, the cone. In particular, the lens and cone are the parts of the thermometer that are visible to the user.

For example, the first end sensor 106L may be a spirometer with an air volume and/or flow sensor and a mouthpiece. In this case, the functional surface 108L comprises the mouthpiece. In particular, the mouthpiece is the part of the spirometer visible to the user.

The first end sensor 106L is positioned at the first end 102L and its functional surface 108L is inscribed in the edge 104L (i.e. the functional surface 108L is integrated within the boundary of the edge 104L). This means that, in a projection along the extension direction X in a transverse plane YZ at the end 102L, the functional surface 108L is positioned inside the edge 104L. Said differently, the projection of functional surface 108L is included in the projection of the side face defined by edge 106L. In other words, the projection of the functional surface 108L lies entirely within the outline of the side face defined by the edge 106L.

Thanks to these features, holding the device 100 is not hindered by the end sensor 106L. In particular, the user can comfortably hold or grip the device 100 by one end, with the hand (e.g. palm) aligned along the extension direction. This feature enhances the device's practicality by also making it compact and easy to store in a pocket, box or similar space.

In this respect, in an embodiment, the functional surface 108L is positioned inside the volume defined by the housing 102 (and therefore by the edge 104L at the end 102L). The end sensor 106L therefore does not protrude out of the housing 102. However, for some sensors, particularly those requiring contact, such as the stethoscope, the functional surface 108L (e.g. the membrane) may protrude up to 5 mm out of the volume defined by the housing 102 along the extension direction X, or even up to 2 mm, or even up to 1 mm.

In an embodiment, the device 100 comprises a second physiological end sensor 106R at the second end 102R. This second physiological sensor 106R is defined similarly to the first physiological sensor 106L and will be referred to as the “second end sensor”.

In an embodiment, shown in FIG. 1, the first end sensor 106L is an electronic stethoscope and the second end sensor 106R is a temperature sensor.

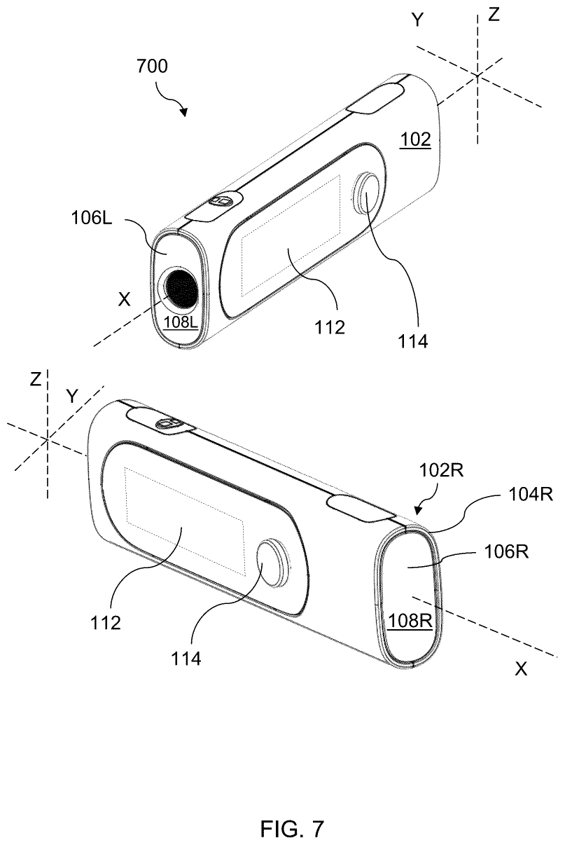

In an embodiment, illustrated in FIGS. 7 to 9 and to be described later, the first end sensor 106L is a temperature sensor and the second end sensor 106R is an electronic stethoscope.

The positioning of the two end sensors 106L, 106R makes it possible to perform these two measurements, in two different positions that will be described in the following paragraphs, with increased maneuverability and a hold that is not hindered by the end sensors 106L, 106R when the user manipulates the device to perform the measurements.

The device 100 further comprises one or more physiological finger sensors 110L, 110R arranged on the housing 102 (hereinafter referred to as “finger sensor”). Each finger sensor 110L, 110R is designed to receive one of the user's fingers (index finger or thumb, for example). Several embodiments will be described below.

The device 100 comprises a display 112, for example a screen (shown dotted in FIG. 1 because the screen contour is invisible to the user, or at least only slightly visible, in this embodiment), intended to display information and/or measurement results to the user.

In an embodiment, the display 112 is configured to display information along a reading direction parallel and/or transverse to extension direction X. In an embodiment, the device comprises a gyrometer and/an accelerometer configured to determine the orientation in space of the device 100 and adapt the reading direction of the display 112 as a function of this orientation. Thus, as will be explained in more detail later, the display may be transverse when the user holds the device with one hand and longitudinal when the user holds the device with two hands.

In an embodiment, the display 112 is positioned on the front face 102F of the housing 102, so that the user can see the display 112 in the navigation position, in the one-handed position and in the two-handed position. A more detailed explanation of the display's placement and its benefits is provided in subsequent sections.

The device 100 further comprises a physical interface 114 with the user, which may take the form of a joystick, arrow, etc. The physical interface 114 is functionally connected to the display 112 and enables the user, for example, to navigate a menu displayed on the display 112 and select actions. The physical interface 114 may be positioned on the 102F front face of the 102 housing.

To simplify navigation, the display 112 and the physical interface 114 are positioned side by side, for example on the front face 102F. In the embodiment shown in FIGS. 1 to 10, the physical interface 114 is positioned on the side of the second end 102R.

FIG. 3 illustrates position 300, the so-called navigation position. The navigation position is, in an embodiment, a one-handed navigation position. This is a position in which the user uses the physical interface 114 to select a measurement. For example, for the illustrated embodiment, the user holds the device 100 in his right or left hand, like a TV remote control, wedging the housing between palm 304 and fingers 306. In view of the arrangement between the display 112 and the physical interface 114, the second end 102R is arranged downwards and the first end 102L is arranged upwards. In this way, the thumb 308 naturally finds the location of the physical interface 114. This position is equally suitable for left- and right-handed users. In this position 300, the display 112 may be configured to display information in a reading direction transverse to the extension direction X. As illustrated here, the menu for selecting available measurements (ECG, SpO2, Stetho, Thermo, for example) is displayed. The user can then navigate and select the measurement he wishes to perform with the physical interface 114.

FIG. 4 illustrates position 400, the so-called one-handed handling position. In particular, this is a one-handed handling position for the first end sensor 106L at the first end 102L. In this position 400, the user holds the device 100 between thumb and forefinger (left or right hand) and brings the functional surface 108L in front of the user's body (which is usually the manipulator too). In the case of the embodiment shown in FIGS. 1 to 6, the first sensor 106L is a stethoscope, the functional surface 108L is the membrane and the body part is the torso 402. The functional surface 108L is then in contact with the body. In this position 400, the display 112 may be configured to display information along a reading direction transverse to the extension direction X. As illustrated here, the information may be a temporal representation of the sounds recorded by the stethoscope, or information relating to the placement of the stethoscope on the body. The user may then view the measurement in progress and adjust the position of the device to improve the volume of the sounds picked up.

FIG. 5 illustrates position 500, the so-called one-handed handling position. In particular, this is a one-handed handling position for the second end sensor 104R at the first end 102R. In this position 400, the user holds the device 100 between thumb and forefinger and brings the functional surface 108R in front of the body. In the embodiment shown in FIG. 1, the second sensor 106R is a thermometer, the functional surface 108R is a lens and/or cone, and the body part is the forehead 502. The device 100 then faces the forehead 502, without contact. Alternatively, the device 100 may come into contact with the front to measure the temperature. In this position 500, the display 112 may be configured to display information along a reading direction transverse to extension direction X. As shown here, the result of the temperature measurement may be displayed.

### The Finger Sensor

As previously mentioned, the device 100 comprises a first finger sensor 110L, positioned near the first end 102L so that in the two-handed holding position the finger sensor 110L is positioned under a finger. Such a position 600 is illustrated in FIG. 6.

This finger sensor 100L may comprise an electrode, for example an ECG electrode and/or an impedance measurement electrode for impedance analysis (e.g. impedance-plethysmogram IPG or body composition), an optical sensor (e.g. PPG, photoplethysmogram, or laser), a force sensor, a pressure sensor, a magnetic sensor, etc.

This first finger sensor 100L may be arranged at different locations on the housing 102.

In relation to FIGS. 1 to 6, the finger sensor 110L is positioned so that the user's index finger 601L, 601R is naturally positioned on it when the device is held by the first end 102L and/or the second end 102R. In this respect, the finger sensor 110L is positioned on the upper face 102To.

According to a non-illustrated embodiment, the finger sensor is positioned on the housing 102 to fall under the thumb when the device is held by the first end 102L and/or the second end 102R. In this respect, the finger sensor is positioned on the front face 102F. For example, the finger sensor may be integrated into the physical interface 114 to simplify the user interface: in FIG. 6, it can be seen that the user may simply extend the thumb to reach the physical interface, or the sensor may be positioned in the vicinity of the physical interface 114.

Alternatively, the finger sensor is positioned on the front face 102Fr, spaced from the physical interface 114.

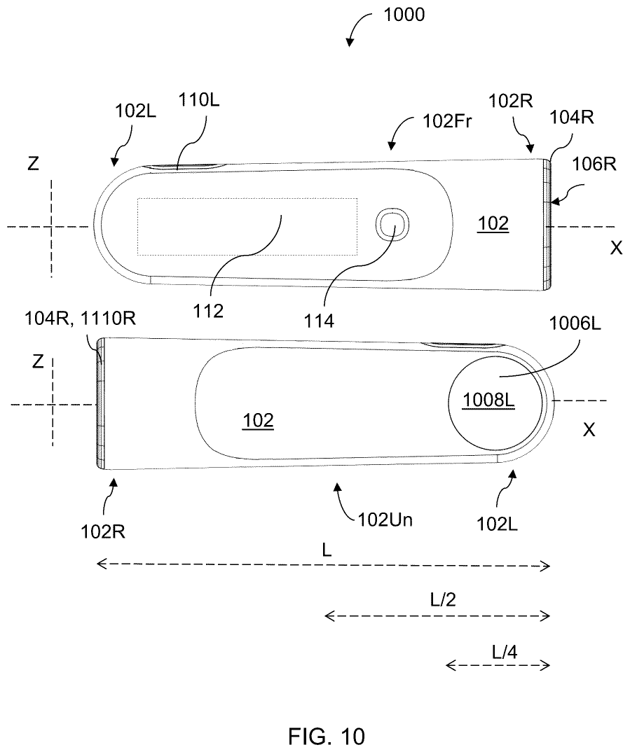

According to an embodiment illustrated in FIG. 10 and which will be described later, the finger sensor is an electrode and the electrode is arranged on all or part of the edge 104L, 104R of the end 102L, 102R, for example via a metal deposit or the addition of a metal part.

In an embodiment, the device 100 comprises a second finger sensor 110R positioned near the second end 102R so that in two-handed handling position 600, each finger sensor 110L, 110R is positioned under a finger.

In an embodiment, “near an end” means “between the edge of the end and strictly half the length L from the edge”. According to another embodiment, “near” means “between the first edge 104L and strictly a quarter of the length L from the first edge 104L”. These examples can be seen in FIG. 2, with distances L/2 and L/4 shown.

In an embodiment, the first finger sensor 110L and the second finger sensor 110R function as electrodes, for example ECG (electrocardiogram) electrodes. The second finger sensor 110L may be positioned similarly to the first finger sensor 110R as described above, i.e. both finger sensors may be located on the upper face 102To (positioning symmetry visible on upper face 102To in FIG. 2), or both finger sensors may be located on the front face 102Fr. More generally, the first finger sensor 110L and the second finger sensor 110R may be aligned parallel to the extension direction X. Symmetrical positioning simplifies the process of taking measurements. Alternatively, one of the two finger sensors 110L, 110R may be placed on the upper face 102To, while the other of the two finger sensors 110L, 110R may be on the front face 102Fr. This arrangement allows the user to perform an ECG by simultaneously touching the two electrodes with fingers from different hands, ensuring ease of use and accurate measurement.

In this embodiment, the two electrodes may be spaced apart, along the extension direction X by a minimum distance of at least 5 cm. This spacing ensures comfortable handling, allowing the user to operate the device without the hands coming into contact with each other.

In an embodiment, only two ECG electrodes may be provided. By dispensing with the third electrode often required for ECG, handling of the device 100 is greatly simplified, since the positioning constraint only applies to the fingers, which fall naturally into position.

FIG. 6 illustrates the two-handed measurement position 600 in which at least one finger sensor 110L, 110R is used. This position consists of holding the device 100 by the ends 102L, 102R, with the hands 602L, 602R respectively. In position 600, the proximal phalanges 604L, 604R of the index fingers 601L, 601R lie in the extension direction X, while the rear face 102R rests on the middle or ring fingers 606L, 606R and the thumbs 608L, 608R rest on the front face 102F.

When the user holds the device 100 in position 600 for two-handed operation, the fingers are naturally positioned on the finger sensors 110L, 110R. In addition, the positioning of the finger sensors also ensures that the display 112 remains clearly visible during manipulation, so that the user can receive information in real time.

In this position 600, the display 112 may be configured to display information along a reading direction parallel to extension direction X. As shown here, a temporal representation of the ECG being measured may be displayed. The user may then view the measurement in progress.

In addition, the finger sensor(s) 110L, 110R, whether on the upper face 102To or the main face 102Fr, are easily reachable in position 600 due to the integration of the end sensor(s) 106L, 106R into the ends 102L, 102R (by being inscribed in the edge). None of the end sensors 106L, 106R require additional (or only marginal) space in relation to the housing 102, and do not interfere with the hand in position 600. The device 100 may therefore be switched quickly and easily between one-handed positions 300, 400, 500 and two-handed position 600.

In an embodiment, the device 100 comprises a third finger sensor 116L positioned near the first end 102L (as illustrated in FIGS. 1 and 2) or the second end 102R so that in the two-handed holding position the third finger sensor 116L is positioned under a finger. For example, the third finger sensor 116L is an optical sensor integrated with the electrode (e.g. an ECG electrode, in order to make ECG and optical measurements at the same time). Such integration enables the same finger position to be used for several measurements.

In an embodiment, the device 100 comprises a fourth finger sensor (not visible because positioned below the optical sensor), similarly located near the first end or the second end. The fourth finger sensor may be a force sensor, which measures a force that the finger applies to the device 100. For example, the force sensor is located under the optical sensor.

In an embodiment, the finger sensor(s) 110L, 110R, 116L are positioned within a volume defined by the housing 102. Put differently, they do not extend outside the housing 102, or only slightly, for example over a distance of less than 2 mm, or, in the case of a finger sensor 110L, 110R, 116L in the physical interface 114, protrude from the physical interface 114. This arrangement means that finger sensors 110L, 110R, 116L do not interfere with handling in positions 300, 400 and 500, where the finger sensor 110L, 110R, 116L is not used.

Thus, the device 100 allows at least two handling positions: a one-handed navigation position 300, one or two one-handed handling positions 400, 500 and a two-handed handling position 600. With two measurement positions, it is thus possible to perform at least three measurements, or even four or five.

### Stethoscope Variant

In the embodiment shown in FIGS. 1 to 6, the display 112 is arranged between the navigation interface 114 and the first sensor 106L, which is a stethoscope (along the extension direction X). Thus, in position 400, i.e. in the one-handed handling position with the first end sensor 106L, which in this case is a stethoscope, the display 112 is close to the body, which may impede reading of the display.

FIGS. 7 to 9 illustrate a device 700, a variant of the device 100 shown in FIG. 1. In device 700, the second end sensor 102R is a stethoscope. The first end sensor 102L may then be a thermometer. The rest of device 700 is unchanged from that shown in FIGS. 1 to 6. In this embodiment, the physical interface 114 is located between the display 112 and the second end sensor 106R, which is a stethoscope (along the extension direction X).

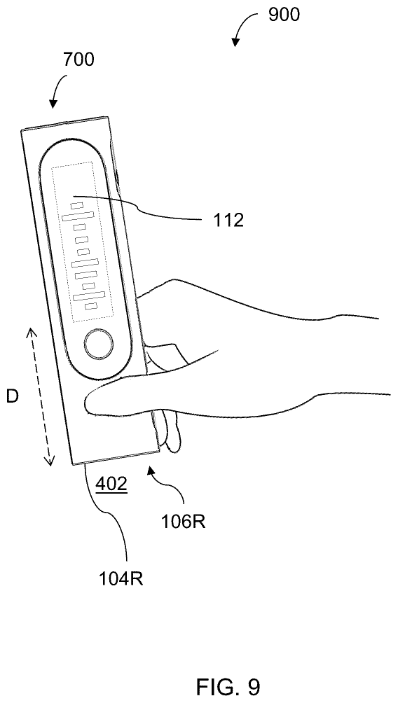

In this way, along the extension direction X, the physical interface 114 is located between the display 112 and the stethoscope 106R (which is the second end sensor 106R). FIG. 9 shows a position 900, corresponding to the position 400 previously described with reference to FIG. 4, in which the stethoscope is used in a one-handed position.

As a result of the positioning of the physical interface 114, the display 112 is further away from the second edge 104R (distance D in FIG. 9 greater than distance D, not shown, in FIG. 4). Thus, when the second sensor 106R (in this case the stethoscope) is in contact with the torso 402, the display 112 is further away from the torso 402 than in the embodiment shown in FIGS. 1 to 6, making it easier to read by limiting the angle at which the neck is bent, and also enabling those with a higher body fat or hair mass to avoid partially masking the display 112.

In particular, when measuring by the stethoscope, the display 112 may indicate instructions to the user for the placement of diaphragm 108R. It is therefore important that the user may easily see the display 112.

In addition, the part of the housing 102 between the second edge 104R and the physical interface 114 allows the device 700 to be held, as illustrated in the navigation position 800, which is identical for this variant.

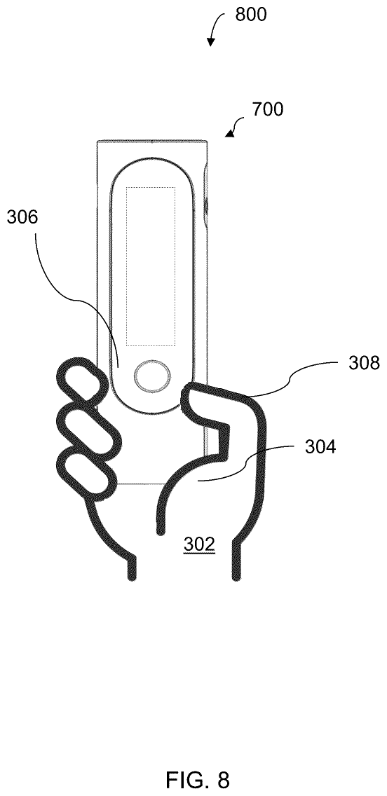

The user thus holds device 700 as in position 800, shown in FIG. 8 (similar to position 300 in FIG. 3). Using the physical interface 114, he selects the stethoscope measurement and then simply moves the stethoscope directly against his torso 402, holding the device 700 between his thumb and forefinger/middle finger, to bring it to position 900 illustrated in FIG. 9. The transition from position 800 to position 900 is particularly simple and does not require the device 700 to be rotated in the hand. In addition, as previously mentioned, the display 112 is more easily visible in this variant thanks to the distance D.

In this variant, when the first end sensor 102L is a thermometer, the position 500 of FIG. 5 may change slightly, insofar as the hand holds the device 700 from the side opposite the thermometer 102L. Consequently, by raising the hand to the front, the device 700 may end up with the face 102Fr facing downwards, but positioning remains simple, with no rotation of the device 700 in the hand.

### Device Variant

FIG. 10 illustrates another embodiment of a device 1000. This embodiment is similar to device 100, with the exception of an end sensor 1006L, which is not inscribed in an edge as previously described. The operating positions 300, 500 and 600 remain unchanged.

In FIG. 10, the first end sensor 1006L is modified, but it could be the second end sensor 1006R.

In this embodiment, a physiological end sensor 1006L comprises a functional face 1008L which is arranged on the front face 102Fr or rear face 102Re. In the illustrated example, functional surface 1008L is positioned on rear face 102Re, on the opposite side to the display 112 and the physical interface 114. In particular, this sensor 1006L is a stethoscope and the functional interface 1008L is a membrane. The membrane typically extends along an orthogonal plane YZ (called the “membrane plane”) the extension direction X

However, the end sensor 1006L remains near the end 1002L, with “near” meaning within half or a quarter of the entire length L of the device 1000 (same definition as above).

The physical interface 114 is located on the front face 102F, between the second end 102R and half the length L, or even a third of the entire length L.

The user may hold the housing 102 between the physical interface 114 and the second edge 104R to apply the membrane 1008L to the torso. In the stethoscope's position of use, the extension direction X is therefore essentially parallel to the torso, meaning it is aligned in a manner that approximates parallelism, allowing for minor deviations due to the natural curvature of the torso or slight variations in the user's grip. By contrast, in position 300 of the device 100, the extension direction X is essentially perpendicular to the torso, indicating that it forms an approximate right angle with the surface of the torso, accounting for minor variations caused by the specific positioning of the device or user anatomy. This terminology, “essentially,” reflects the practical realities of use where perfect geometric alignment is not strictly required, as long as the intended functionality and measurement accuracy are not compromised.

FIG. 10 illustrates a further embodiment of the previously mentioned finger sensor. This embodiment is not directly related to the stethoscope described above.

Indeed, one or more finger sensors 1010R (only finger sensor 1010R is concerned in FIG. 10, but alternatively both finger sensors 1010R, 1010L may be concerned) may be positioned on all or part of the edge 104R of the end 102R. In this way, contact is no longer made solely by the finger, but also by the palm of the hand, in the two-handed handling position, as in position 600.

The sensor may be made by depositing metal on the edge or by adding a metal part.

This method is particularly suitable when the finger sensor is an electrode. In the case of an optical sensor, positioning under the finger is still preferable.

### Variants

As described, combinations are possible between the given variants and modes of realization.

In particular, the first end sensor as described may be at the second end and, where appropriate, the second end sensor as described may be at the first end.

### Presentation of the Device and its Environment

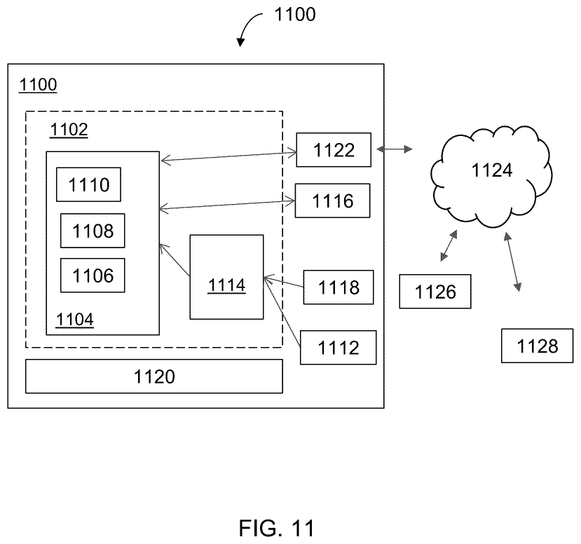

FIG. 11 shows a schematic diagram of the architecture of a device 100, 700, 1000 (referenced 1100 on this figure) as described and its environment.

The device 1100 comprises a control unit 1102 with control circuitry 1104 including a processor 1106, memory 1008 and I/O (In/Out) interface 1110 for communicating with other components.

Memory 1008 stores programs, instructions or other items that enable navigation on the device 1100 and measurements to be taken (algorithms in particular). In particular, the memory 1008 breaks down into a volatile memory, of the RAM type, and a non-volatile memory, of the flash (or ROM or SSD) type.

The device 1100 comprises one or more sensors 1112 (all the sensors described above are shown under a single reference 1112).

The control unit 1102 typically comprises an interface module 1114 interfacing between the sensors 1112 and the I/O interface 1110 of the control circuitry 1104. The interface module 1114 comprises notably ADCs, filters, amplifiers, etc.

The device 1100 further comprises a display 1116, which communicates with the I/O interface 1110, and a physical interface 1118, which communicates with the interface module 1114 for menu navigation of the display 1116.

To supply the various components with electrical power, the device 1100 comprises a battery 1120, for example a rechargeable battery. The battery 1120 is configured to power the control unit 1104, the display 1114 and the sensors 1112.

Finally, for connectivity, the device 1100 comprises a wireless communication module 1122 (BLUETOOTH® (a short-range wireless technology standard), BLE (BLUETOOTH® low energy), BLE, Wifi, cellular, etc.), connected to the control circuitry 1102. The module 1122 enables communication via a communications network 1124 with a mobile terminal 1126 (e.g. a smartphone-type cell phone) and/or a remote server 1128. The physiological data thus acquired by device 1100 may be stored, analyzed, processed in the server 1128 and displayed by the mobile terminal 1126. The mobile terminal 1126 may also act as a relay between the device 1100 and the server 1128 (e.g. in the case of BLUETOOTH® or BLE communication).

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements and components of compositions, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A portable physiological measurement device holdable by a user, comprising
a housing of elongated shape along an extension direction and comprising, along the extension direction, a first end defining a first edge and a second end defining a second edge,
a first physiological end sensor positioned at the first end and comprising a functional surface intended to be positioned facing a user, the functional surface being inscribed in the edge at the end,
a second physiological end sensor positioned at the second end, and
a physiological finger sensor arranged on the housing near the first end or the second end.

2. The portable physiological measurement device of claim 1, wherein the second end defines a second edge and the second physiological sensor comprises a functional surface inscribed in the second edge.

3. The portable physiological measurement device of claim 1, wherein the housing comprises an essentially parallelepiped shape with a front face and a rear face arranged between the first end and the second end, wherein the second end physiological sensor is arranged on the rear face, near the second end.

4. The portable physiological measurement device of claim 1, wherein a distance along the extension direction between the first end and the second end is of length L, and wherein a distance that separates the physiological finger sensor from the first end or the second end is less than half the length L.

5. The portable physiological measurement device of claim 4, wherein the distance that separates the physiological finger sensor from the first end or the second end is less than a quarter of the length L.

6. The portable physiological measurement device of claim 1, wherein the physiological end sensor is positioned within a volume defined by the housing or at most 1 mm outside the volume defined by the housing.

7. The portable physiological measurement device of claim 1, wherein:
the physiological end sensor is an audio sensor and the functional surface comprises a membrane, or
the physiological end sensor is a temperature sensor and the functional surface comprises a cone, or
the physiological end sensor is a spirometer and the functional surface comprises a mouthpiece.

8. The portable physiological measurement device of claim 1, further comprising a display, wherein:
in a one-handed handling position for the first end sensor, the display is configured to display information along a reading direction transverse to the extension direction,
in a two-handed handling position in which the finger sensor is used, the display is configured to display information along a reading direction parallel to the extension direction.

9. The portable physiological measurement device of claim 1, wherein the finger sensor comprises an optical sensor.

10. The portable physiological measurement device of claim 1, wherein the physiological finger sensor is a first physiological finger sensor arranged near the first end and the device further comprises a second physiological finger sensor, arranged on the housing near the second end.

11. The portable physiological measurement device of claim 1, wherein each physiological finger sensor comprises an electrode.

12. The portable physiological measurement device of claim 1, wherein the first physiological finger sensor and the second physiological finger sensor are aligned parallel to the extension direction.

13. The portable physiological measurement device of claim 1, comprising a physical interface arranged on the housing and the second physiological finger sensor is positioned on the physical interface.

14. The portable physiological measurement device of claim 1, wherein the housing comprises an essentially parallelepiped shape with a front face and a top face arranged between the first end and the second end, the front face and the top face being connected.

15. The portable physiological measurement device of claim 14, wherein the first physiological finger sensor and the second physiological finger sensor are positioned on the top face.

16. The portable physiological measurement device of claim 14, comprising a display, the display being located on the front face.

17. The portable physiological measurement device of claim 16, comprising a physical interface positioned on the front face, the physical interface being positioned between the second edge, respectively the first edge, and half, or even a third, of a length of the housing along the direction of extension from the second edge, respectively the first edge.

18. The portable physiological measurement device of claim 1, comprising a display and comprising a gyrometer and/an accelerometer configured to determine the orientation in space of the device and to adapt the reading direction of the display as a function of this orientation.

19. The portable physiological measurement device of claim 1, wherein the finger sensor is positioned inside the volume defined by the housing or at most 2 mm outside the volume defined by the housing.

20. A method of using the device of claim 1, comprising:
measuring the physiological end sensor by the user in a one-handed handling position, and
measuring with the finger physiological sensor by the user in a two-handed handling position.
